(19) United States
(12) Patent Application Publication
(10) Pub. No.: US 2019/0254624 A1
(43) Pub. Date: Aug. 22, 2019

(54) TISSUE CHARACTERIZATION WITH ACOUSTIC WAVE TOMOSYNTHESIS

(71) Applicants: The United States of America, as represented by the Secretary, Department of Health and Human Serv, Bethesda, MD (US); The Johns Hopkins University, Baltimore, MD (US)

(72) Inventors: Bradford Wood, Potomac, MD (US); Reza Seifabadi, Baltimore, MD (US); Fereshteh Aalamifar, Baltimore, MD (US); Emad Boctor, Baltimore, MD (US); Arman Rahmim, Baltimore, MD (US)

(73) Assignees: The United States of America, as represented by the Secretary, Department of Health and Human Serv, Bethesda, MD (US); The Johns Hopkins University, Baltimore, MD (US)

(21) Appl. No.: 16/307,925

(22) PCT Filed: Jun. 8, 2017
(86) PCT No.: PCT/US2017/036599

§ 371 Date: Dec. 6, 2018

Related U.S. Application Data
(60) Provisional application No. 62/347,437, filed on Jun. 8, 2016.

(51) Int. Cl. A61B 8/12; A61B 8/00; A61B 90/50

(52) U.S. Cl. CPC A61B 8/12; A61B 8/4218; A61B 8/15; A61B 8/4477; A61B 8/4263; A61B 90/50

(57) ABSTRACT

Imaging of internal structure of a patient, such as the prostate, is performed using ultrasound tomography by inserting a first ultrasound probe into the rectum of the patient, positioning a second ultrasound probe on an abdomen of the patient, and aligning the first and second ultrasound probes with one another to obtain acoustic information for reconstructing tomographic images of the internal structure. Light sources can also be shined to the tissue of interest, such as prostate say by a transurethral catheter thus making photoacoustic waves that can be received by the said TRUS or TRAB/TRPR transducers to reconstruct photoacoustic tomographic image of the tissue, as well.

### CROSS REFERENCE TO RELATED APPLICATIONS

This application claims the benefit of U.S. Provisional Application No. 62/347,437, filed Jun. 8, 2016, which is herein incorporated by reference in its entirety.

### FIELD OF THE DISCLOSURE

The present disclosure is directed to ultrasound imaging, and more specifically, to limited angle transmission acoustic wave tomography, also referred to as acoustic wave tomosynthesis. The acoustic wave can be generated mechanically by an US transducer, e.g., ultrasound tomosunthesis (USTS), or photoacoustically by shining a light source, e.g., photoacoustic tomosynthesis (PATS).

### BACKGROUND

Effective diagnostic imaging of internal anatomy is critical to detection of cancers and other disease. For example, prostate cancer is the most common male cancer in the United States with an estimated 220,000 new cases and 28,000 deaths in 2015. A key to survival is early detection of cancer. Systematic sextant biopsies under transrectal ultrasound (TRUS) guidance have been the gold standard method since 1989. TRUS is real-time, relatively low cost, and shows the prostate capsule and boundaries. However, it suffers from poor spatial resolution and low sensitivity for cancer detection (40-60%). Typically, six biopsies are obtained in a regular but random fashion. This is somewhat blind in which instead of directing the needle to a specific target, it is placed in a specific geographic region of the prostate.

Although MRI is typically a superior imaging modality for visualizing the prostate gland, nerve bundles, and cancer lesions, it is not typically a real-time imaging modality and the cost of in-gantry prostate biopsy is significantly higher making it impractical. Fusion of TRUS and multi parametric MRI (mpMRI) can allow benefiting from both imaging modalities. In fusion guided-biopsy, targeting information is solely dependent on MR images. Even though US-MRI fusion guided biopsy has shown to be highly sensitive to detect higher-grade cancer, it still suffers from false positives for lower-grade cancers resulting in unnecessary biopsies. Another limitation is that it still requires MR imaging which is the most expensive imaging modality. Also, MRI is still less accessible to rural and suburb areas. Therefore, an ultrasound only based prostate biopsy technique has been a clinical need for decades. Some US based technologies have recently been proposed to address this clinical need, including elastography, Doppler, and US tissue characterization. Although several studies reported improvement in prostate cancer identification with quasi-static elastography, there are still some limitations in reproducibility, subjectivity, and the inability of this method to differentiate cancer from chronic prostatitis. Time series analysis is an interesting new machine learning technique to perform the tissue characterization and has recently shown some promising results for marking cancerous areas of prostate using the US RF image. This method is still based on a post-processing of reflection data and the reproducibility of the results are questionable.

### SUMMARY OF THE DISCLOSURE

As discussed herein, methods and systems for aligning ultrasound probes that can transmit and receive ultrasound signals are provided for achieving tomographic imaging of internal structures of the body, such as the prostate. Such systems and methods can provide, for example, improved accuracy and efficiency in cancer diagnosis.

In one embodiment, a method of imaging an internal structure of a patient using limited angle ultrasound tomography includes inserting a first ultrasound probe into the rectum of the patient, positioning a second ultrasound probe on an abdomen or perineum of the patient, aligning the first and second ultrasound probes with one another, transmitting and receiving ultrasound signals via the first and second ultrasound probes, and reconstructing tomographic images based on the ultrasound signals received by the first and second ultrasound probes.

In another embodiment, a transurethral ultrasound probe can be placed in addition to the TRUS probe to make tomographic image of the bottom half of the prostate.

In another embodiment, a transurethral ultrasound probe can be placed in addition to the TRAB/TRPR probe to make tomographic image of the top half of the prostate.

In one embodiment, the acoustic wave is generated by the transmitting US probe. In another embodiment, the acoustic wave is generated by shining light to the tissue of interested via photoacoustic phenomenon and both of the mentioned probes can act as receiver to reconstruct the tomographic image.

The tomographic images can be reconstructed by determining acoustic properties in each pixel of the tomographic image, such as the speed of sound (SOS) or attenuation in USTS scenario, or optical properties such as optical absorption coefficient in PATS. The acoustic properties can be calculated by determining a distance between a transmitting ultrasound probe and a receiving ultrasound probe, determining a measured travel time between a respective transmitting ultrasound probe and a respective receiving ultrasound probe.

The ultrasound probes can be of various types. For example, in one embodiment the first ultrasound probe comprises a bi-plane transrectal ultrasound probe and the second ultrasound probe comprises a linear array transducer. The first ultrasound probe can have a linear transducer array or a curved transducer array.

The manner in which the probes are moved and controlled can vary. In one embodiment, the first and second ultrasound probes are coupled to one or more robotic arm and can be repositioned using the robotic arm. The first ultrasound probe can also have a tracked passive or motorized brachytherapy stepper to facilitate repositioning, or one or more force sensors to restrict the amount of force applied to the patient by the first ultrasound probe.

In other embodiments it can include a transrectal ultrasound probe, a linear array transducer, and one or more mechanical arms coupled to the rectal ultrasound probe and linear array transducer to mechanically constrain movement of the rectal ultrasound probe and linear array transducer and facilitate alignment of them relative to one another. The transrectal ultrasound probe can comprise a bi-plane ultrasound probe, with a linear sagittal or curved axial transducer array or a tri-planar TRUS probe with an angled linear array, a curved axial and a curved sagittal array or a combination of these. The one or more mechanical arms can be robotically controlled arms that are configured to align the transrectal ultrasound probe and the linear array transducer.

The foregoing and other features and advantages of the invention will become more apparent from the following detailed description of several embodiments which proceeds with reference to the accompanying figures.

### BRIEF DESCRIPTION OF THE FIGURES

FIG. 1A shows a schematic of a full angle approach, known as ultrasound computed tomography (USCT) and FIG. 1B shows a schematic of a partial angle approach, also referred to herein as ultrasound tomosynthesis (USTS).

FIG. 2 illustrates a novel method of performing USTS for prostate cancer diagnosis and screening.

FIG. 3 illustrates an exemplary setup for controlling the movement of the abdominal probe.

FIG. 4 illustrates an exemplary setup for performing ex vivo testing of the systems and methods disclosed herein.

FIG. 5A illustrates a 3D printed mold for comparing with MRI-histology information.

FIG. 5B illustrates a 3D printed box for creating a US friendly mold.

FIG. 5C illustrates the US friendly patient specific mold and an ex vivo prostate tissue right after surgery for USTS-MR-histology correspondence.

FIG. 6 illustrates an exemplary USTS ex vivo setup.

FIG. 7 illustrates raw data showing that some of the waveforms contained electrical noise, or refracted delayed signals which could result in miss-selection of the time of flight.

FIGS. 8A-C show the simulation results, with FIG. 8A showing the groundtruth simulation phantom, FIG. 8B showing a reconstructed velocity map using conjugate gradient (Diff-CG), and FIG. 8C showing expectation maximization (Diff-EM) methods.

FIG. 9A shows a B-mode of a slice of the mock prostate made of the patient-specific mold with plastisol as lesion and water as prostate, and FIG. 9B shows a reconstruction image using an expectation maximization method.

FIGS. 10A-D show images relating to a real ex vivo prostate, including a B-mode of a slice of the prostate (FIG. 10B) and a corresponding MRI slice showing three lesions (FIG. 10D).

FIG. 11 illustrates the use of transurethral, transabdominal, and transrectal ultrasound transducers.

FIG. 12A illustrates a light source that includes an array of light elements (e.g., LEDs) on a delivery member (e.g., a Foley catheter).

FIG. 12B illustrates a light source that includes multiple arrays of light elements (e.g., LEDs) on a delivery member (e.g., a Foley catheter).

FIG. 12C illustrates a cross section view of the light source of FIG. 12B.

FIG. 13 illustrates a photoacoustic system with a TRUS probe, a TRAB probe, and a light source.

FIGS. 14A and 14B illustrate transvaginal and transabdominal acoustic wave tomography systems that can be used alone or in combination with each other and/or other devices.

FIGS. 15A and 15B illustrate full photoacoustic tomographic reconstruction systems utilizing a 3D probe placed perennially, a TRUS probe, and/or a light source delivered adjacent a lesion.

### DETAILED DESCRIPTION OF SEVERAL EMBODIMENTS

The following description is exemplary in nature and is not intended to limit the scope, applicability, or configuration of the invention in any way. Various changes to the described embodiment may be made in the function and arrangement of the elements described herein without departing from the scope of the invention.

As used in this application and in the claims, the singular forms “a,” “an,” and “the” include the plural forms unless the context clearly dictates otherwise. Additionally, the term “includes” means “comprises.” Further, the terms “coupled” or “associated” generally mean electrically, electromagnetically, and/or physically (e.g., mechanically or chemically) coupled or linked and does not exclude the presence of intermediate elements between the coupled or associated items absent specific contrary language.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

US tomographic imaging uses two transducers and works based on transmission, rather than reflection in B-mode, in which the transmitter and receiver transducers are located at different known positions with respect to the volume of interest. The received signal can be used to reconstruct the volume's acoustic properties such as speed of sound (SOS), attenuation, and spectral scattering maps. This information, which is not available on current US machines, can be used to differentiate among different tissue types including abnormal tissues. In some embodiments, software can be provided that receives data from the transmitter and receiver probes as inputs, and uses that raw data to reconstruct a SOS/attenuation map for each pixel in addition to the B-mode image.

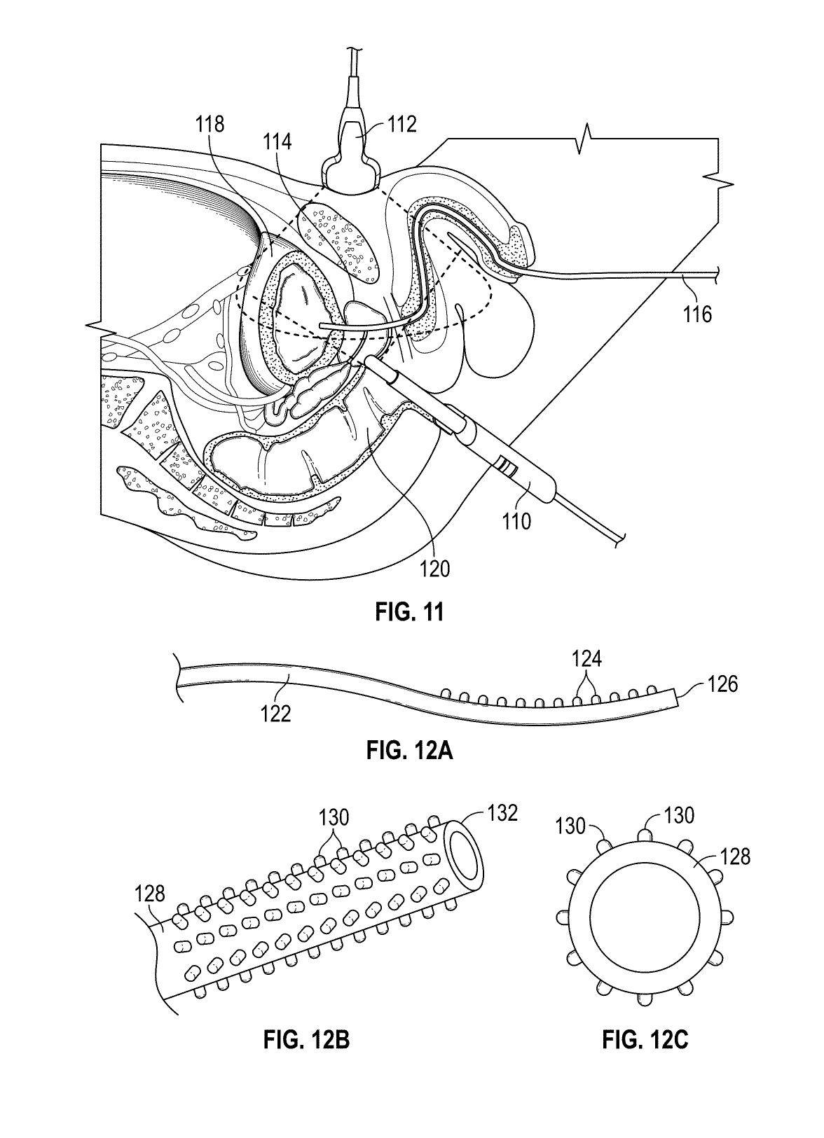

In addition to a transrectal ultrasound (TRUS) probe, which is can be used during biopsy for B-mode imaging, the systems and methods disclosed herein include at least one additional (e.g., a second) transducer, such as a transabdominal (TRAB) and/or a transperineal (TRPR) transducer on the patient. The later can be positioned with or without a robot arm that aligns (e.g., autonomously) the TRAB and/or TRPR probe to the TRUS in order to make a tomography image. An embodiment with multiple probes is shown, for example, in FIG. 11.

Transmission ultrasound can be performed using full 180 degree angle or limited angle techniques. For limited angle ultrasound, the angle depends on the range of motion and is less than 180 degrees. For example, assuming the axial distance between the probes is 10 cm and two linear probes of length 6 cm are used (with no movement), the angle will be 73 degrees (=2*a sin(0.6)). Therefore, at an angle of 180 degrees, the probes would be infinitely long.

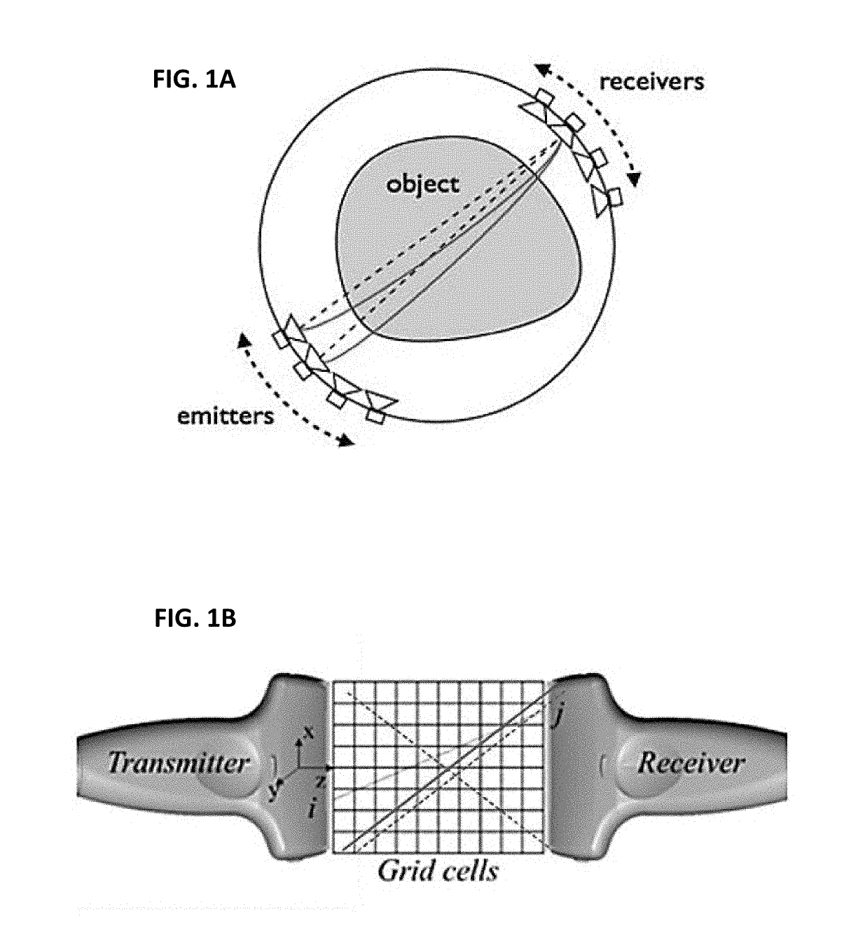

FIG. 1A shows a schematic of a full angle approach, known as ultrasound computed tomography (USCT) and FIG. 1B shows a schematic of a partial angle approach, also referred to herein as ultrasound tomosynthesis (USTS).

In the former case, transmitter and receivers move physically or electronically 180 degrees whereas in latter, transmitter and receiver move a limited angle, where the angle depends on the distance of transducers or the number of transmitters and receivers. USCT can be used for breast imaging and imaging extremities and USTS can be used for breast imaging and imaging bones/limbs. The methods and systems described herein provide the ability to extend tomography imaging to prostate.

USTS provides tissue acoustic properties such as the speed of sound and attenuation, in “each pixel” or region of interest thus can detect cancerous areas based on the fact that cancerous and non-cancerous prostate tissue have different acoustic properties. For example, since cancerous tissue has a different speed of sound and attenuation than healthy tissue, they can be demonstrated with different colors in velocity/attenuation maps, even though they may look similar in standard B-mode US.

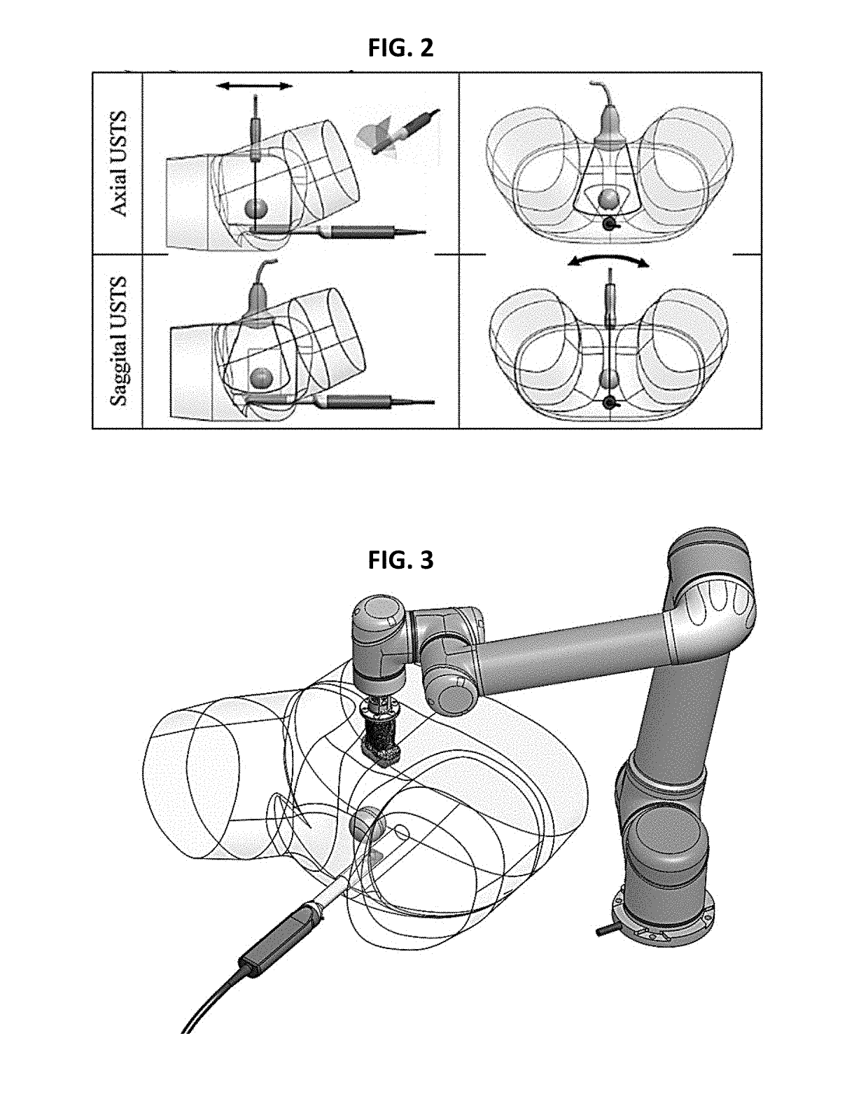

FIG. 2 illustrates a novel system and method of performing USTS for prostate cancer diagnosis and screening. As shown in FIG. 2, the system can comprise the use of at least two ultrasound devices, such as a bi-plane TRUS probe positioned in the rectum and a linear array transducer positioned on the patient's abdomen above the bladder. The top row of FIG. 2 shows the use of a curved array of a bi-plane TRUS probe, and the bottom row of FIG. 2 shows the use of a linear array of a bi-plane probe for USTS reconstruction. The abdominal probe can be manipulated either manually or with a robotic arm, such as in the directions indicated by the arrows of FIG. 2. In the manual scenario, both TRUS and abdominal probes can be mechanically constrained and aligned. In the robotic scenario, the abdominal probe can be configured to align itself with the TRUS probe. The TRUS probe can be positioned with a tracked passive or motorized brachytherapy stepper. FIG. 3 illustrates an exemplary setup for controlling the movement of the abdominal probe. The assisted movement device shown in FIG. 3 can be motorized or support manual movement, and provides more precise control of the abdominal probe.

Image registration of two or more imaging modalities as described herein can be performed in various manners to align the data from the different sensors (e.g., different US probes) and utilize a common coordinate system.

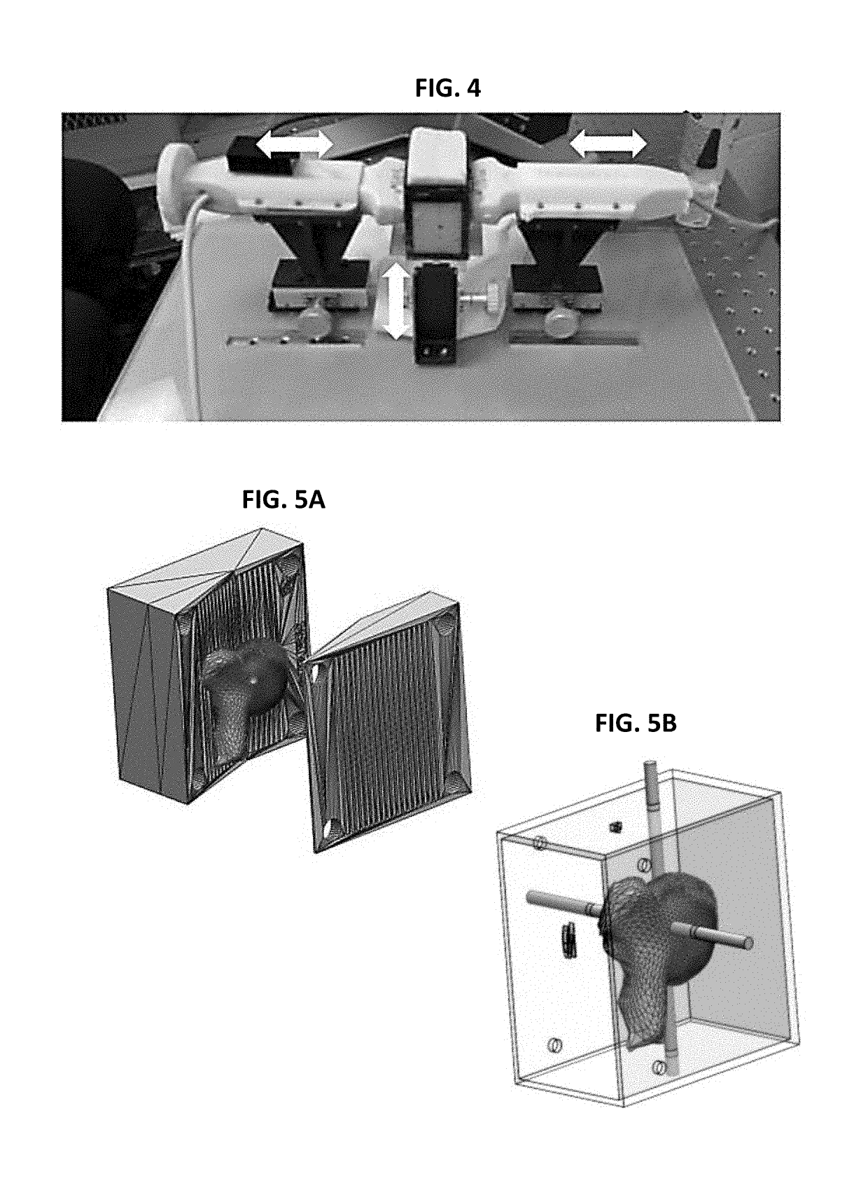

Methods and systems for performing prostate USTS are described in more detail below, including details of testing performed for ex vivo prostate USTS to illustrate its effectiveness. The testing discussed herein used a mock prostate and lesions with comparable speed of sound. The setup of the ex vivo testing is depicted in FIG. 4. Two linear US probes (128 arrays, 6 cm) were aligned. The distance between the probes was adjustable to provide sufficient contact against the scanned volume. The ex vivo prostate was put inside a patient specific, US friendly mold. In the example shown in FIG. 4, the mold was placed inside a container with transparent rubber windows and a small amount of liquid was injected to fill the gaps between the prostate, mold, and container. The container was placed between the aligned probes, and provided so that its height can be adjusted in order to scan different slices.

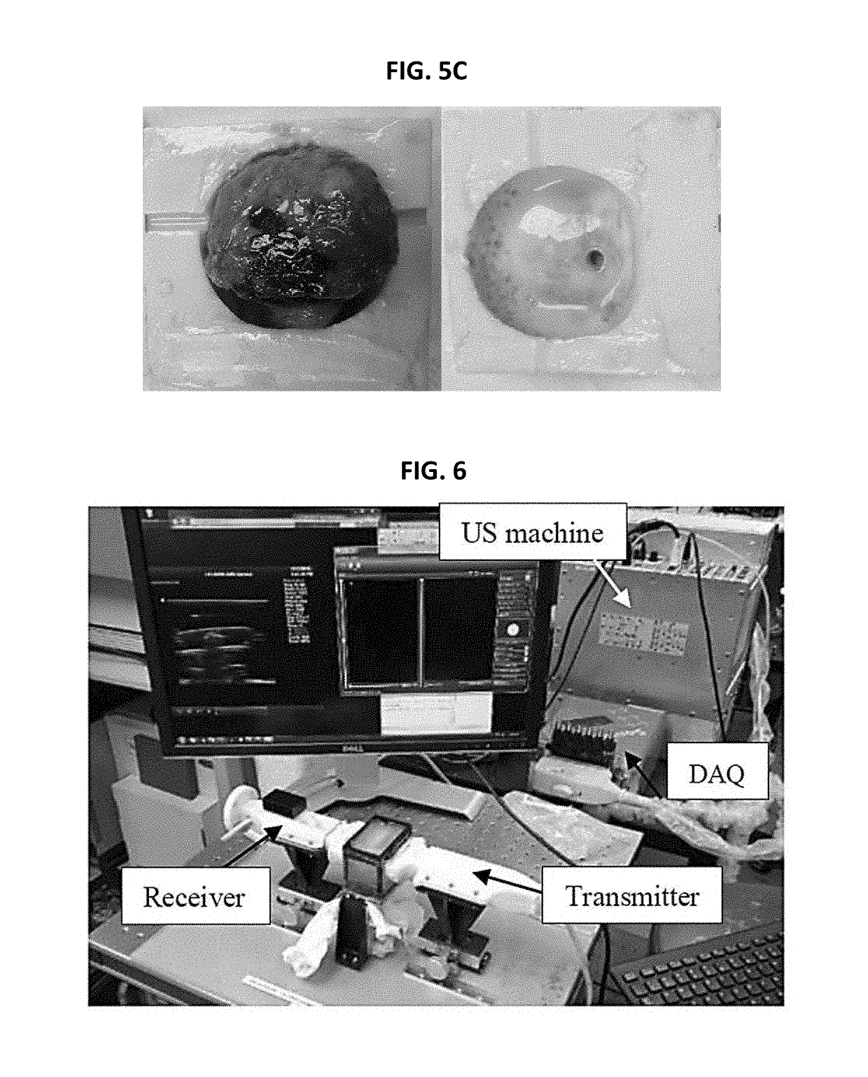

To facilitate comparison of the USTS image reconstructed using this setup with MRI image and histology, in one embodiment the following technique was performed. First, a patient specific mold (as shown in FIG. 5A) with grooves to guide a histology knife was 3D printed. The grooves were 3 mm apart and result in histology slices corresponding to MR image slices. Second, the same mold was created using an US friendly material with marks indicating the corresponding slices to be scanned using the US probes. The US friendly mold was made of acrylamide with 1523 m/s speed of sound. This material does not decay, is rigid enough to hold the prostate, and has appropriate speed of sound suitable for reconstruction. In order to make the mold, first, the prostate (with seminal vesicles) was segmented from the patient's MR image. This prostate is saved as a .stl file and printed using a 3D printer (uprint, Stratasys). The 3D printed prostate was positioned inside a box at similar position and orientation compared to MRI 3D printed mold using guide rods as shown in FIG. 5B and then, the acrylamide was poured into the box. After solidification, the rods were removed and the mold was cut such that the 3D printed prostate can be removed. FIG. 5C shows the US friendly mold.

The prostate was put inside the mold cavity and the mold's halves glued together. Then, the mold was inserted into a container. The container holds the mold in place during the USTS scan, can be filled with liquid to fill the gaps between mold and prostate, and provides windows made of Mylar sheet to provide US transparency. The container was marked with lines that determine the slices that correspond to the MRI slices.

Two linear arrays can be used (e.g., two linear array Ultrasonix probes). In one example, the transmitting probe was connected to an Ultrasonix Sonixtoch scanner (Vancouver, BC). The receiving probe was connected to an Ultrasonix Data Acquisition (DAQ) device which can receive the US waveforms of 128 channels in parallel with sampling frequency of 40 MHz. The DAQ device was connected to the US machine using a USB cable to transfer the received data. In addition, the trigger-out of the US machine was set to produce the line trigger (i.e., send a trigger pulse after each transmission) and was connected to the trigger-in of the DAQ device using a BNC cable to synchronize the transmit-receive sequences. FIG. 6 shows the overall USTS ex vivo setup.

To reconstruct an USTS image, i.e. to calculate the speed of sound in each pixel of the image (FIG. 1B), two pieces of information are used: the accurate distances between each transmit-receive pair, and the measured travel time between them.

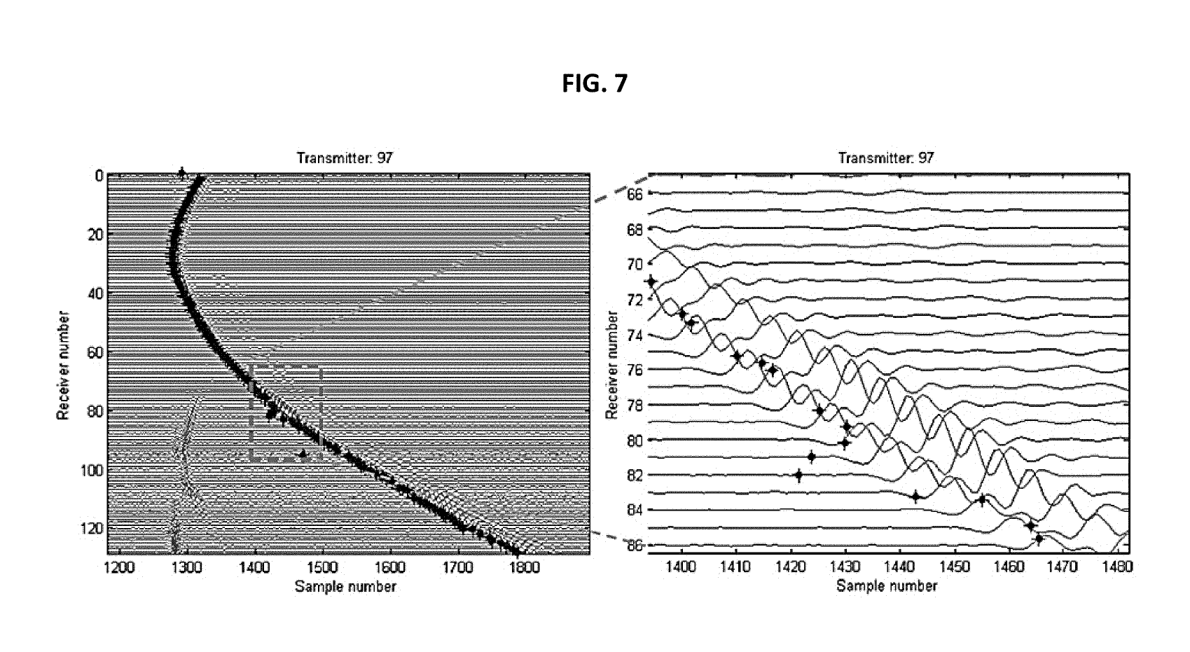

The US data collected contains of 128 waveforms per transmitter, each corresponding to one receiver and one image (slice), calculated from 128 transmissions. Hence, in order to compute the speed of sound, the time of flight should be picked at all 128×128 (=16384) waveforms. A MATLAB interface was implemented to pick the travel times semi-automatically. The initial locations of the time of flights were estimated using a center of mass method over an estimated window as:

$${t}_{c m} = \frac{{\int }_{t} {ts}^{2} ( t ) dt}{{\int }_{t} {s}^{2} ( t ) dt} + {t}_{bg} - w \qquad ( 1 )$$

where s(t) is the intensity of the received signal at time t. s(t) is set to zero outside [tbg−w, tbg+w], where tbg is the estimated background time of flight, w is half of a certain window length to reduce the effect of noise and refractions. As shown in FIG. 7, some of the waveforms contained electrical noise, or refracted delayed signals which could result in miss-selection of the time of flight. The MATLAB interface allows the user to correct for these miss-selections.

The grid area between transmit-receive pairs (FIG. 1B) is formulated as a system matrix and the following equation can be used to find the image based on straight-ray US propagation approximation:

S(X−X_{bg})=T−T_{bg} (2)

where S is the system matrix, X is a vectored concatenation of the image matrix, and T is a vector containing the time of flight measurements. Xbg and Tbg are the known background speed of sound values, and the measured time of flights for background respectively. The background is collected by scanning a slice that only contains of the acrylamide gel. This information can be helpful in compensating for probe misalignment and measurement bias. Various methods can be used to solve for this equation, such as the expectation maximization algorithm which is suitable for limited data reconstruction.

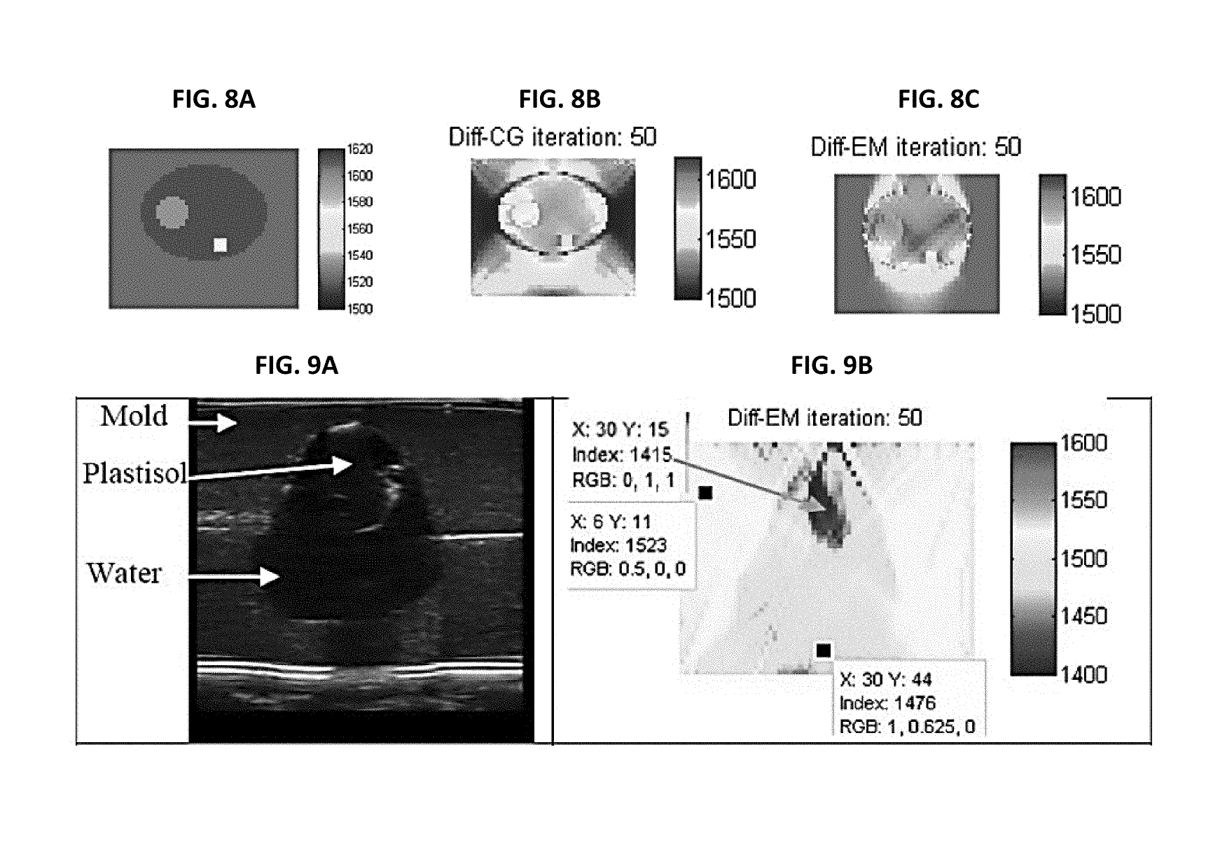

The example simulated the mathematics of the reconstruction problem without considering US wave propagation properties. The ground-truth image was created based on the typical of size of the prostate and lesions. As shown in FIG. 8, the prostate can be modeled as a 3×4 cm ellipse and contains two lesions of size 5 and 10 mm in diameter. The speeds of sound in prostate were set to 1614 m/s for prostate region and, 1572 m/s and 1596 m/s for the two lesions.

Using the setup shown in FIG. 6, an image of the mock prostate was acquired. The US machine was set in B-mode image acquisition mode with 7 cm depth, 5 MHz US frequency, and aperture size equal to 1 to enable sequential transmission of US waves. A mock ex vivo study was performed by filling the mold cavity with water (1490 m/s) and attaching to the inner part of the mold a lesion made of plastisol (1300 m/s). The container with the mold inside was put between the aligned probes and their axial distance was adjusted to 50 mm. US gel was applied to the probe tips to enhance the coupling and the center slice was chosen to do USTS data collection.

A simulation phantom was created in MATLAB based on the prostate description given above.

Two methods of solving for equation (2) were used (conjugate gradient and expectation maximization). It was observed that a background speed of 1523 m/s produced a better image than ones with 1300 m/s and 1010 m/s, corresponding to plastisol and silicon ecoflex respectively. Artifacts in the images are due to the limited angle data but the lesions are still distinguishable from the prostate. FIGS. 8A-C show the simulation results, with FIG. 8A showing the groundtruth simulation phantom, FIG. 8B showing a reconstructed velocity map using conjugate gradient (Diff-CG), and FIG. 8C showing expectation maximization (Diff-EM) methods.

FIG. 9A shows a B-mode of a slice of the mock prostate made of the patient-specific mold with plastisol as lesion and water as prostate. After time of flight picking using the semi-automatic MATLAB-based interface, the image was reconstructed. The expectation maximization method could produce better results and is shown in FIG. 9B. The theoretical speeds of sound are around 1523, 1480, and 1375 m/s for mold, water, and plastisol respectively and as shown in the figure, these values in one pixel in each of these areas are estimated as 1523, 1476, and 1415 m/s.

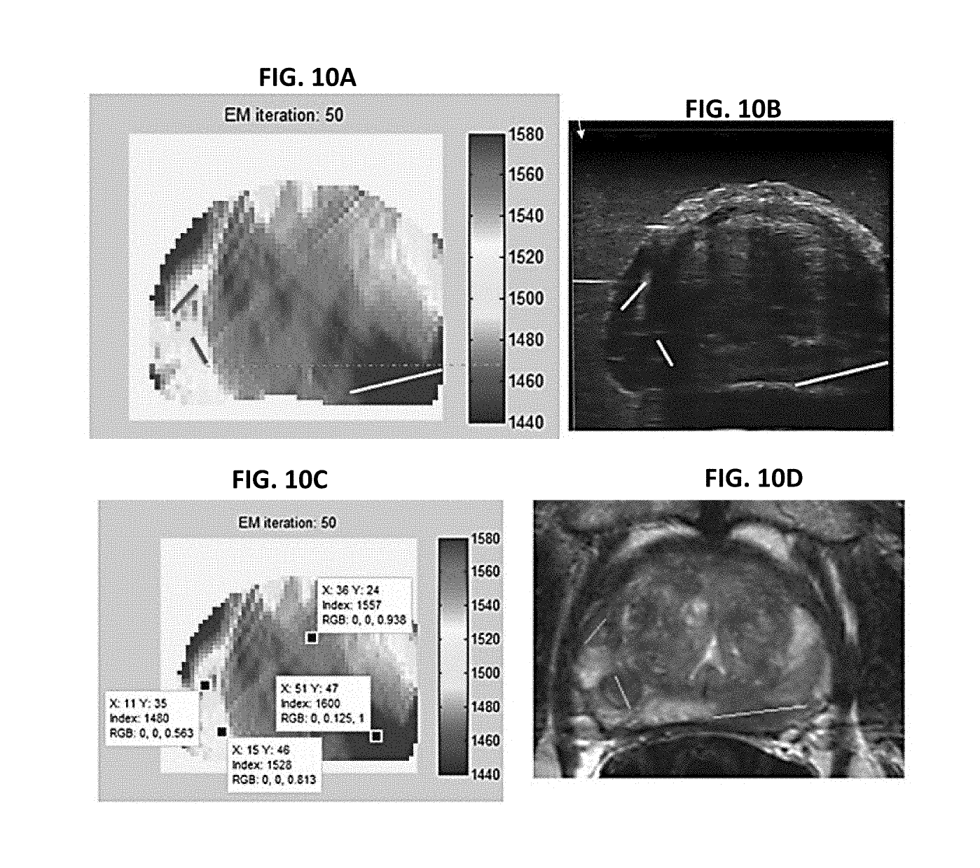

FIG. 10B shows a B-mode of a slice of the real ex vivo prostate and is compared to a corresponding MRI slice showing three lesions shown in FIG. 10D. The tumor in the right is malignant while the other two tumors in left are benign. From the images on the right, it is evident that the tomographic SOS image can detect and differentiate these lesions, apparently because they have different SOS compared to the healthy tissues.

In some embodiments, the systems and methods described herein can include a TRAB and/or TRUS probe with a longer array than is typical for such probes. In other embodiment, the TRUS or TRAB/TRPR imaging arrays can be virtually extended by moving the probe using the robot. In other embodiments, safety features can be provided with the system to ensure the amount of force applied to the patient by a probe does not exceed certain predetermined thresholds. For example, one or more sensors (e.g., strain/stress sensors) can be positioned on the probes and configured to communicate information to the user about an amount of force applied by the probe on the patient.

The systems and methods disclosed herein permit prostate imaging with high sensitivity and specificity without substantially altering the current clinical workflow. The tomographic images produced by the techniques disclosed herein can provide quantitative images, thus increasing sensitivity and specificity of US-based prostate cancer screening. These systems and methods can reduce health disparity by reducing the cost of imaging, reduce the need for additional trips to the hospital, and enhance clinical outcomes.

In some embodiments, another ultrasound probe, such as a transurethral ultrasound probe can be placed in addition to the first and/or second US probe to make tomographic image of other regions of the tissue of interest, such as the bottom portion or the top portion of the prostate. FIG. 11 illustrates such a system and method of operation.

As shown in FIG. 11, a transrectal probe 110 can be inserted into a rectum 120 of a patient and a transabdominal probe 112 can be placed adjacent the skin of the patient to image the same area (i.e., a prostate 114) as the transrectal probe 110.

In some embodiments, another ultrasound probe may be used in combination with either the first or second ultrasound probe. In some embodiments, the third ultrasound probe may be a transurethral probe. FIG. 11 illustrates a transurethral US transducer 116 inserted through a urethra of the patient to a bladder area 118. In other embodiments, the transurethral device can comprise one or more light sources as described below.

The first, second or third ultrasound probe may have an embedded source for electrometric emission that is capable of generating specific wavelengths or patterns of wavelengths. The source of electrometric emission may be a light source in the infrared, visible or ultraviolet spectrum. The light source may include any incandescent, LED, laser, source or any other source known in the art that is capable of generating photo-acoustic waves (based on known photoacoustic phenomenon) in the tissue of interest, such as prostate tissue.

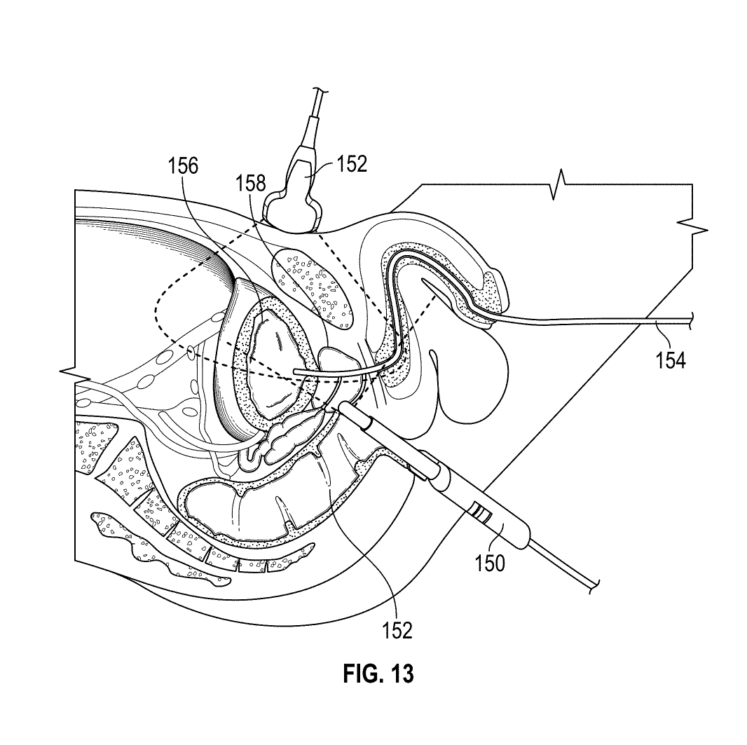

FIGS. 12A-12C illustrate catheters (e.g., Foley catheters) that carry one or more light elements (e.g., one or more arrays of LEDs) in the vicinity of a distal end of the catheter. The light elements can be positioned on one side, or partially or completely surround the catheter. FIG. 12A illustrates an embodiment in which a catheter 122 has an array of light elements 124 (e.g., LEDs) along one side of the catheter at its distal end 126. FIGS. 12B and 12C illustrate an embodiment in which a catheter 128 has a plurality of arrays of light elements 130 (e.g., LEDs) that surround the distal end 132 of the catheter. Thus, the arrangement of the light elements can vary from a small angle of electrometric emission to 360 degrees of electrometric emission.

FIG. 13 illustrates a photoacoustic system with a TRUS probe 150 (inserted into a rectum 152) and a TRAB probe 152 (positioned adjacent an external area of the patient), and a light source 154 positioned at an internal area of the patient. As discussed herein, in one embodiment, the light source can comprise light elements positioned on a distal end 154 of a catheter (e.g., a Foley catheter) delivered to a bladder region 156 of the patient to facilitate imaging of adjacent internal structures (such as the prostate 158) as shown in FIG. 13.

When the light energy is delivered to the biological tissue, it gets partly absorbed by the tissue and converts to heat energy leading to expansion in the tissue. This expansion causes mechanical movements that creates acoustic wave and can be detected by an ultrasonic transducer. The amplitude of the transmitted acoustic wave by each part of the tissue is a function of its optical absorption coefficient. Hence the received signal can be used to reconstruct an image representing the tissue optical absorption which can classify normal, benign, and malignant tissue.

For the purposes herein, transrectal or trans-urethral light delivery with TRUS probes as a receiver for prostate photoacoustic imaging can be performed in a variety of manners consistent with the teachings herein. See, e.g., Valluru, K., Chinni, B., Bhatt, S., Dogra, V., Rao, N. and Akata, D., 2010, July, Probe design for photoacoustic imaging of prostate in Imaging Systems and Techniques (IST), 2010 IEEE International Conference on (pp. 121-124). IEEE, and Bell, M. A. L., Guo, X., Song, D. Y. and Boctor, E. M., 2015, Transurethral light delivery for prostate photoacoustic imaging, Journal of biomedical optics, 20(3), pp. 036002-036002, both of which are incorporated by reference herein.

Utilizing one ultrasound probe as receiver, a method of delay and sum beamforming can be used to reconstruct the photoacoustic image which contains artifacts and blurring effects due to data incompleteness and inaccuracy of the method. With the embodiment proposed here, since two probes are used as receivers, more accurate photoacoustic tomographic image reconstruction becomes possible.

As noted above, the source of electrometric emission may be arrayed around the probe in 360 degrees, or some lesser degree of array (e.g., 45-180 degrees, 90-180 degrees, or 180-270 degrees) to focus or diffuse the light source, wherein the probe can be rotated to generate acoustic waves at different angles.

In still other embodiments the first, second or third ultrasound probe may have reflective or refractive surface materials such as a metallic coating or reflective polymers. In some embodiments the photo-acoustic waves generated from the tissue of interest are received by a US transducer to reconstruct a photoacoustic tomographic image. In other embodiments the US transducers receiving the photo-acoustic waves generated from the tissue are b TRUS or TRAB/TRPR transducers. The photoacoustic image can show different optical properties of scanned tissues such as optical absorption coefficient. Since different tissues have different optical properties, they show up differently in such image making another layer of information for tissue classification and prostate cancer screening.

In some embodiments, the light source can be attached to a TRUS probe, a TRAB/TRPR probe, or other suitable structures to excite the tissue and generate photoacoustic waves as disclosed herein.

The light source can be any suitable light source for the functions and purposes disclosed herein, including, for example, laser and LED light sources.

In some embodiment, a TRUS probe and a TRAB or a drop-in US probe can be used as transmitter and receiver, respectively during robot-assisted prostatectomy or partial nephrectomy procedures (e.g. using da Vinci robot, Intuitive Surgical, Sunnyvale, Calif.). In such scenario, both transmitter and receiver, or at least the TRAB or the drop-in probe can be manipulated using one of the arms of the surgical robot.

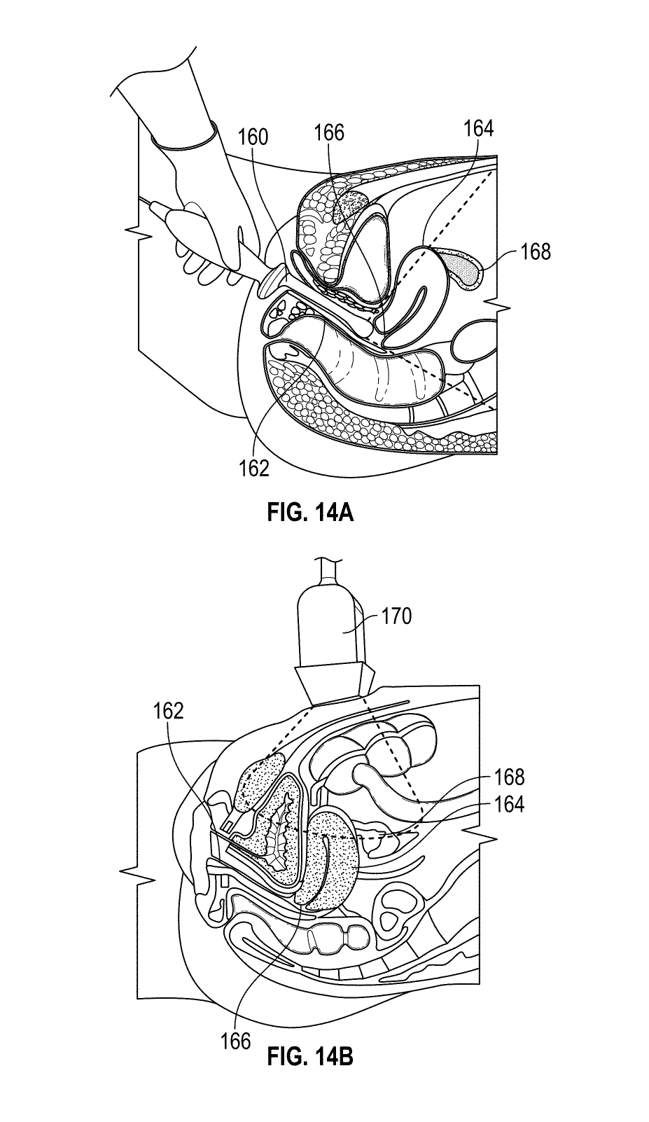

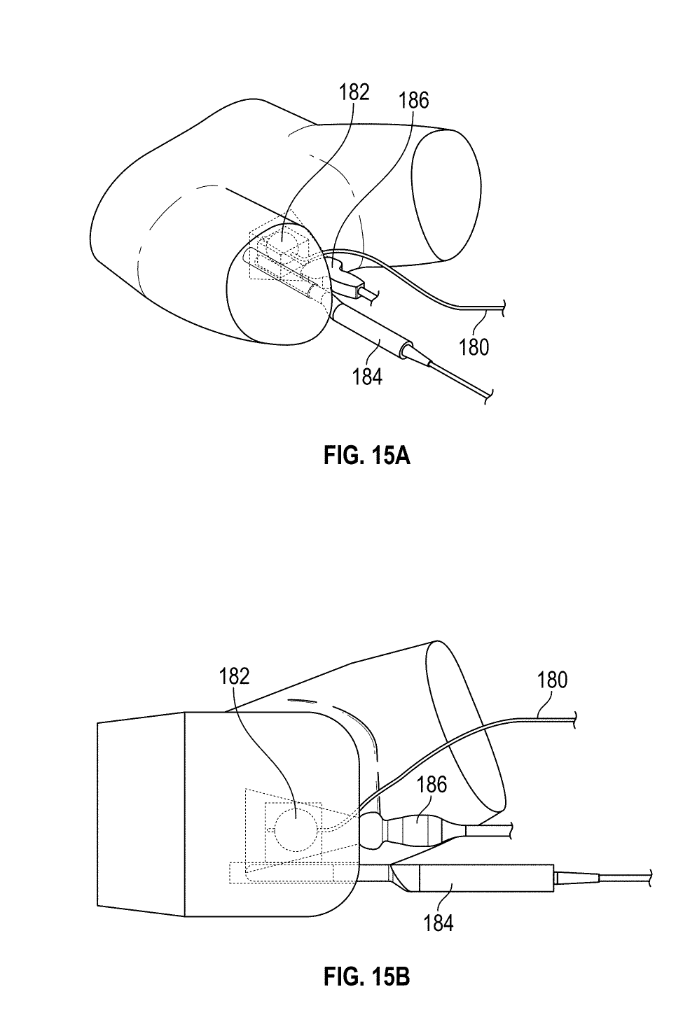

Depending on the area to be imaged, different internal approaches can be taken for at least one of the probes. For example, in some embodiments, the first US probe is an esophageal ultrasound transducer and a second US transducer from outside the body are aligned to reconstruct a tomography image to detect esophageal cancer. In other embodiments, similar TRUS and TRAB/TRPR probes are used for bladder cancer screening. In some embodiments, a transurethral probe and a TRAB/TRPR US probes are aligned to make a tomographic image of the bladder. Other approaches can include transvaginal acoustic wave tomographic systems (FIGS. 14A and 14B) and photoacoustic systems that use combinations of perineal probes and TRUS probes (FIGS. 15A and 15B).

In some embodiments, the first US probe is an intravascular ultrasound transducer (iVUS) and the second probe is outside body and aligned to make tomographic images (either acoustic or photoacoustic) of the vessels for different purposes.

In some embodiments, two external US transducers are aligned to make a tomographic image in order to verify plaque in carotid artery.

FIG. 14A illustrates a transvaginal probe 160 which can be inserted across or through the vagina 162 to image, for example, a woman's uterus 164, cervix 166, ovaries 168, and pelvic area generally. FIG. 14B illustrates a transabdominal probe 170 which can positioned externally to also image, for example, a woman's uterus 164, cervix 166, ovaries 168, and pelvic area generally.

Thus, FIGS. 14A and 14B collectively illustrate an embodiment in which vaginal and transabdominal imaging modalities are used. For convenience, FIGS. 14A and 14B illustrate these modalities being used separately; however, based on the disclosure herein, it should be understood that the two modalities can be aligned for concurrent use as described herein in the various embodiments for tissue characterization of different structures including but not limited to bladder, uterus and ovaries.

In some embodiment, a TRUS and a TPUS 2D/3D probe can be used with one or more light source to provide near to full angle photoacoustic US tomosynthesis of the prostate. FIGS. 15A and 15B show this concept.

FIGS. 15A and 15B illustrate a light source 180 (e.g., a plurality of light elements positioned on a distal end of a catheter) positioned adjacent a desired internal area of the patient to be imaged (i.e., a prostate 182). A transrectal probe 184 and a transperineal probe 186 can be positioned as shown in FIGS. 15A and 15B to provide improved imaging of the internal area.

In some embodiments, at least one US probe of the system is an endoscopic probe, or an intraductal probe.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method of imaging an internal structure of a patient using acoustic wave tomography, comprising:
inserting a first acoustic wave probe into an orifice of the patient and into an internal cavity of the patient;
positioning a second acoustic wave probe adjacent to or within the patient;
generating an acoustic wave with the second acoustic wave device and directing it at the internal structure of the patient;
aligning the first and second acoustic wave probes with one another;
transmitting and receiving acoustic signals via the first and second ultrasound probes; and
reconstructing tomographic images based on the acoustic signals received by the first and second acoustic wave probes.

2. The method of claim 1, wherein the acoustic wave signals comprise ultrasound signals.

3. The method of claim 1, wherein the acoustic wave signals comprise light generated by at least one of the first and second acoustic wave probes.

4. The method of claim 1, wherein the internal cavity is a rectum of the patient.

5. The method of claim 1, wherein the internal cavity is a vagina of the patient.

6. A method of imaging an internal structure of a patient using ultrasound tomography, comprising:
inserting a first ultrasound probe into the rectum of the patient;
positioning a second ultrasound probe on an abdomen of the patient or on his perineum;
aligning the first and second ultrasound probes with one another;
transmitting and receiving ultrasound signals via the first and second ultrasound probes; and
reconstructing tomographic images based on the ultrasound signals received by the first and second ultrasound probes.

7. The method of claim 6, wherein the tomographic images are reconstructed by determining acoustic properties in each pixel of the tomographic image.

8. The method of claim 7, wherein the determined acoustic properties in each pixel of the tomographic image comprises the speed of sound and/or attenuation in each pixel of the image, wherein the act of determining the acoustic properties comprises determining a distance between a transmitting ultrasound probe and a receiving ultrasound probe, and determining a measured travel time between a respective transmitting ultrasound probe and a respective receiving ultrasound probe.

9. The method of claim 6, wherein the first ultrasound probe comprises a bi-plane tri-plane, or a 3D transrectal ultrasound probe and the second ultrasound probe comprises a linear, curved, or 3D array transducer.

10. The method of claim 9, wherein the first ultrasound probe comprises a linear transducer array.

11. The method of claim 9, wherein the first ultrasound probe comprises a curved transducer array.

12. The method of claim 9, wherein the first ultrasound probe comprises a 3D transducer array.

13. The method of claim 6, wherein one or both of the first and second ultrasound probes are coupled to one or more robotic arm, and wherein the act of aligning the first and second ultrasound probes comprises moving one or both of the first and second ultrasound probes via the robotic arm.

14. The method of claim 6, wherein the act of aligning the first and second ultrasound probes comprises repositioning the first ultrasound probe using a tracked passive or motorized brachytherapy stepper.

15. The method of claim 6, further comprising receiving force information from one or more force sensors on the first ultrasound probe to restrict the amount of force applied to the patient by the first ultrasound probe.

16-39. (canceled)

40. A method of imaging an internal structure of a patient comprising:
inserting a transvaginal probe into the patient;
aligning a transabdominal probe for tissue characterization of the internal structure of the patient;
transmitting and receiving acoustic signals; and
reconstructing the internal structure using the received acoustic signals.

41. The method of claim 40, wherein the internal structure comprises at least one of the bladder, uterus, or ovaries.

42-43. (canceled)
